(19) United States
(12) Patent Application Publication
(10) Pub. No.: US 2009/0297577 A1
(43) Pub. Date: Dec. 3, 2009

(54) Local Delivery of Apolipoproteins and Their Derivatives

(75) Inventors: Josiah Wilcox, Santa Rosa, CA (US); Ayala Hezi-Yamit, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/131,643

(22) Filed: Jun. 2, 2008

(51) Int. Cl. A61F 2/04; A61K 38/17; A61K 9/00

(52) U.S. Cl. 424/423; 514/12; 424/484

(57) ABSTRACT

Disclosed are medical devices and methods for the local delivery and treatment of vascular conditions. The methods and treatments involve local delivery of at least one apolipoprotein. The vascular conditions described herein include plaque rupture, aneurysm, stenosis, restenosis, atherosclerosis, ischemic myocardial infarct and combinations thereof.

### FIELD OF THE INVENTION

The present invention relates to the local delivery of apolipoproteins and their derivatives for the treatment of stenosis, restenosis, atherosclerosis, ischemic myocardial infarct, aneurism, restinosis and atherosclerotic plaque rupture.

### BACKGROUND OF THE INVENTION

Cardiovascular disease is a leading cause of morbidity and mortality. Recently, it has been discovered that high density lipoprotein (HDL) has beneficial therapeutic effects such as reduction of cardiovascular disease. It has been shown that infusion of synthetic apolipoproteins facilitate HDL effects, specifically, HDL mediated cholesterol efflux from arterial walls and enhancing cholesterol clearance.

Once such apolipoprotein is apolipoprotein A-I (ApoA I), which has been shown, by systemic infusion, to increased cholesterol efflux with production of small HDLs, enhanced bile acid and neutral sterol excretion. A variant of ApoA I is apolipoprotein A-I Milano (ApoA IM), which is a naturally occurring variant of ApoA I, wherein the arginine at position 173 is replaced with a cysteine. The variant was discovered in a small group of people in Italy and has been shown to be linked to improved arterial protection and low cardiovascular risk.

However, to date, studies have focused on the systemic delivery of apolipoproteins wherein, more of the compound is needed to attain a therapeutic dosage. When a compound is delivered systemically, there is an increased risk of toxic side effects, and the half-life and bioavailability of the compound are restrictive of the benefit.

As a result, to reduce the problems associated with systemic delivery of a apolipoprotein to treat cardiovascular disease, medical devices and methods of local delivery need to be devised which address these problems.

### SUMMARY OF THE INVENTION

Disclosed herein are medical devices and methods for the local delivery of a bioactive agent and for the treatment of cardiovascular conditions. The methods and treatments involve local delivery of at least one apolipoprotein or derivative thereof. Vascular conditions include, but are not limited to, plaque rupture, aneurysm, stenosis, restenosis, atherosclerosis, ischemic myocardial infarct and combinations thereof.

In one embodiment, a medical device system is described for localized treatment of a cardiovascular condition comprising: a medical device; a polymer matrix associated with the medical device; and a therapeutically effective amount of an apolipoprotein, the apolipoprotein residing within the polymer matrix.

In another embodiment, the apolipoprotein is ApoA-I Millano. In one embodiment, the ApoA-I Millano is present at a weight of 1 μg to 1000 μg. In another embodiment, the apolipoprotien is ApoA-I peptide mimetic. In yet another embodiment, the ApoA-I peptide mimetic is present at a weight of 1 μg to 1000 μg.

In one embodiment, the medical device is selected from the groups consisting of stents, catheters, micro-particles, probes, and vascular grafts. In another embodiment, the cardiovascular condition is selected from the group consisting of plaque rupture, aneurysm, stenosis, restenosis, atherosclerosis, ischemic myocardial infarct, and combinations thereof.

In one embodiment, the polymer matrix is biodegradable. In another embodiment, the polymer matrix comprises polymers selected from the group consisting of poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes and biomolecules such as fibrin, fibrinogen, cellulose, polysaccharides or carbohydrates (i.e. starch, hyaluronic acids, dextran, heparin sulfate, chondoritin sulfate, heparin, alginate), proteins (i.e. polyamino alcohols, polyphosphazines, polyanhidrides), collagen, and combinations thereof. In one embodiment, the medical device comprises a ratio of polymer to apolipoprotein, wherein the ratio is between about 1:1 to about 1:20.

In one embodiment, a method is described for localized treatment of a cardiovascular condition comprising: a) providing a medical device comprising a polymer matrix, wherein said polymer matrix comprises a therapeutically effective amount of an apolipoprotein, said apolipoprotein; b) implanting said medical device within a blood vessel; and c) allowing said medical device to locally deliver said apolipoprotein thereby treating said cardiovascular condition.

In another embodiment, the apolipoprotein is ApoA-I Millano. In one embodiment, the ApoA-I Millano is present at a weight of 1 μg to 1000 μg. In another embodiment, the apolipoprotien is ApoA-I peptide mimetic. In yet another embodiment, the ApoA-I peptide mimetic is present at a weight of 1 μg to 1000 μg.

In one embodiment, the medical device is selected from the groups consisting of stents, catheters, micro-particles, probes, and vascular grafts. In another embodiment, the cardiovascular condition is selected from the group consisting of plaque rupture, aneurysm, stenosis, restenosis, atherosclerosis, ischemic myocardial infarct, and combinations thereof.

In one embodiment, the polymer matrix is biodegradable. In another embodiment, the polymer matrix comprises polymers selected from the group consisting of poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes and biomolecules such as fibrin, fibrinogen, cellulose, polysaccharides or carbohydrates (i.e. starch, hyaluronic acids, dextran, heparin sulfate, chondoritin sulfate, heparin, alginate), proteins (i.e. polyamino alcohols, polyphosphazines, polyanhidrides), collagen, and combinations thereof. In one embodiment, the medical device comprises a ratio of polymer to apolipoprotein, wherein the ratio is between about 1:1 to about 1:20.

### DEFINITION OF TERMS

Bioactive Agent: As used herein “bioactive agent” shall include any drug, pharmaceutical compound or molecule having a therapeutic effect in an animal. Exemplary, non-limiting examples include anti-proliferatives including, but not limited to, macrolide antibiotics including FKBP 12 binding compounds, estrogens, chaperone inhibitors, protease inhibitors, protein-tyrosine kinase inhibitors, leptomycin B, peroxisome proliferator-activated receptor gamma ligands (PPARy), hypothemycin, nitric oxide, bisphosphonates, epidermal growth factor inhibitors, mTOR inhibitors, antibodies, proteasome inhibitors, antibiotics, anti-inflammatories, anti-sense nucleotides, and transforming nucleic acids. Bioactive agents can also include cytostatic compounds, chemotherapeutic agents, analgesics, statins, nucleic acids, polypeptides, growth factors, and delivery vectors including, but not limited to, recombinant micro-organisms, and liposomes.

Exemplary FKBP 12 binding compounds include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid) and zotarolimus (ABT-578). Additionally, and other rapamycin hydroxyesters may be used in combination with the terpolymers of the present invention.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Biodegradable: As used herein “biodegradable” refers to a polymeric composition that is biocompatible and subject to being broken down in vivo through the action of normal biochemical pathways. From time-to-time bioresorbable and biodegradable may be used interchangeably, however they are not coextensive. Biodegradable polymers may or may not be reabsorbed into surrounding tissues, however, all bioresorbable polymers are considered biodegradable. Biodegradable polymers are capable of being cleaved into biocompatible byproducts through chemical- or enzyme-catalyzed hydrolysis.

Nonbiodegradable: As used herein “nonbiodegradable” refers to a polymeric composition that is biocompatible and not subject to being broken down in vivo through the action of normal biochemical pathways.

Not Substantially Toxic: As used herein “not substantially toxic” shall mean systemic or localized toxicity wherein the benefit to the recipient is out-weighted by the physiologically harmful effects of the treatment as determined by physicians and pharmacologists having ordinary skill in the art of toxicity.

Pharmaceutically Acceptable: As used herein “pharmaceutically acceptable” refers to all derivatives and salts that are not substantially toxic at effective levels in vivo.

### DETAILED DESCRIPTION OF THE INVENTION

Described herein are medical devices and methods for local delivery of an apolipoprotein. Local delivery of an apolipoprotein or other therapeutic agent can provide many benefits over systemic delivery including, but not limited to, a smaller therapeutic compound requirement, the compound action is delivered directly to the target lesion, a decreased risk of systemic toxic side effects, a decreased half-life of the therapeutic agent and bio-availability are not restrictive of the benefit. As such, local delivery of an appropriate apolipoprotein can aid in the treatment of, among other conditions, restenosis, stenosis, ischemic myocardial infarct, atherosclerosis, stabilization of vulnerable plaque, and combinations thereof.

There are many different apolipoproteins that are known in the art including, but not limited to, ApoA I, ApoA II, ApoA IV, ApoA V, ApoB 48, ApoB 100, ApoC I, ApoC II, ApoC III, and ApoC IV. In particular, ApoA, specifically type I (ApoA I), is known to be useful in the treatment of cardiovascular disease by aiding in the elimination of cholesterol from arteries. ApoA I is known to be a major component of HDL, also known as “good cholesterol.” ApoA IM is a naturally occurring variant of ApoA I which was discovered in 1980 and was traced to a single 18^{th} century family in Italy. The decedents carrying the variant have lowered levels of HDL with no increased risk of cardiovascular disease, which is why ApoA IM is thought to have a greater effect in treating cardiovascular disease than its non-variant counterpart.

In one embodiment, the apolipoprotein can be ApoA I. In another embodiment, the apolipoprotein can be the naturally occurring variant of ApoA I, ApoA IM. Variations, mimetics, and mutations of these apolipoproteins are within the scope of the present description.

Described herein are medical devices and methods useful for local delivery to vascular areas in mammals susceptible to, or effected by aneurysm, atherosclerosis, plaque rupture, ischemic myocardial infarct, stenosis and/or restenosis. Local, site specific delivery of apolipoproteins can enhance cholesterol clearance and aid in plaque stabilization. Local delivery can also prolong the beneficial effects of the apolipoprotein within the treated vessel and minimize systemic exposure to the drug. The main benefits of local delivery of an apolipoprotein would be comprised of local treatment of aneurysm, atherosclerosis, plaque rupture, ischemic myocardial infarct, stenosis and/or restenosis.

The apolipoprotein used for local delivery can be any endogenous or synthetic apolipoprotein. In some embodiments, the apolipoprotein can be any derivative, prodrug, small peptide fragment of, or combination thereof, of the apolipoprotein.

It will be understood by those skilled in the art, that ApoA I and ApoA IM are but two of many pharmaceutically acceptable apolipoproteins. Many other pharmaceutically acceptable forms can be synthesized and are still considered to be within the scope of the present description. Moreover, many derivatives are also possible that do not affect the efficacy or mechanism of action of the apolipoproteins and are commonly referred to as mimetics. Therefore, the present description is intended to encompass ApoA I, ApoA IM, and pharmaceutically acceptable derivatives, prodrugs, mimetics, small peptide fragments having biological activity, and combinations thereof.

The apolipoproteins discussed herein may be added to implantable medical devices. The apolipoprotein may be incorporated into the polymer coating applied to the surface of a medical device or may be incorporated into the polymer used to form the medical device. The apolipoprotein may be coated to the surface with or without a polymer using methods including, but not limited to, precipitation, coacervation, and crystallization. The apolipoprotein also may be bound to the surface of the polymer or medical device covalently, ionically, or through other intramolecular interactions, including without limitation, hydrogen bonding and Van der Waals forces.

The medical devices used may be permanent medical implants, temporary implants, or removable devices. For example, and not intended as a limitation, the medical devices may include stents, catheters, micro-particles, probes, and vascular grafts.

In one embodiment, stents may be used as a drug delivery platform. The stents may be vascular stents, urethral stents, biliary stents, or stents intended for use in other ducts and organ lumens. Vascular stents, for example, may be used in peripheral, neurological, or coronary applications. The stents may be rigid expandable stents or pliable self-expanding stents. Any biocompatible material may be used to fabricate stents, including, without limitation, metals and polymers. The stents may also be bioresorbable. In one embodiment, vascular stents are implanted into coronary arteries immediately following angioplasty. In another embodiment, vascular stents are implanted into the abdominal aorta to treat an abdominal aneurysm.

In one embodiment, metallic vascular stents are coated with one or more apolipoproteins, the compounds of ApoA I or ApoA IM. The apolipoprotein may be dissolved or suspended in any carrier compound that provides a stable, un-reactive environment for the apolipoprotein. The stent can be coated with a apolipoprotein coating according to any technique known to those skilled in the art of medical device manufacturing. Suitable, non-limiting examples include impregnation, spraying, brushing, dipping and rolling. After the apolipoprotein is applied to the stent, it is dried leaving behind a stable apolipoprotein delivering medical device. Drying techniques include, but are not limited to, heated forced air, cooled forced air, vacuum drying or static evaporation. Moreover, the medical device, specifically a metallic vascular stent, can be fabricated having grooves or wells in its surface that serve as receptacles or reservoirs for the apolipoproteins described herein.

The effective amount of apolipoprotein used can be determined by a titration process. Titration is accomplished by preparing a series of stent sets. Each stent set will be coated, or contain different dosages of apolipoprotein. The highest concentration used will be partially based on the known toxicology of the compound. The maximum amount of drug delivered by the stents will fall below known toxic levels. The dosage selected for further studies will be the minimum dose required to achieve the desired clinical outcome. In one embodiment, the desired clinical outcome is defined as a site specific enhanced removal of cholesterol. In another embodiment, the desired clinical outcome is defined as a decreased instance of ischemic myocardial infarction, plaque rupture, stenosis, restenosis, or combination thereof.

In another embodiment, the apolipoprotein is precipitated or crystallized on or within the stent. In yet another embodiment, the apolipoprotein is mixed or associated with a suitable biocompatible polymer (bioerodable, bioresorbable, or non-erodable). In yet another embodiment, the apolipoprotein is associated with a polymer matrix. The apolipoprotein can be dispersed in the polymer matrix, can be ionically bound to the polymer matrix, can be physically attracted to the polymer matrix, or can be dispersed on top of the polymer matrix.

The polymer-apolipoprotein blend can then be used to produce a medical device such as, but not limited to, stents, grafts, micro-particles, sutures and probes. Furthermore, the polymer-apolipoprotein blend can be used to form controlled-release coatings for medical device surfaces. For example, and not intended as a limitation, the medical device can be immersed in the polymer-apolipoprotein blend, the polymer-apolipoprotein blend can be sprayed, or the polymer-apolipoprotein blend can be brushed onto the medical device. In another embodiment, the polymer-apolipoprotein blend can be used to fabricate fibers or strands that are embedded into the medical device or used to wrap the medical device.

In one embodiment, the polymer chosen must be a polymer that is biocompatible and minimizes irritation to the vessel wall when the medical device is implanted. The polymer may be either a biostable or a bioabsorbable polymer depending on the desired rate of release or the desired degree of polymer stability. Bioabsorbable polymers that can be used include poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, polly sacharides or carbohydrates (i.e. starch, hyaluronic acids, dextran, heparin sulfate, chondoritin sulfate, heparin, alginate), proteins (i.e. polyamino alcohols, polyphosphazines, polyanhidrides), and collagen.

The above polymers can be used to form a polymer matrix. The polymer matrix can be a hydrophobic matrix or can be a hydrophilic matrix. In one embodiment, the polymer matrix can comprise a combination of both hydrophobic and hydrophilic regions.

Also, biostable polymers with a relatively low chronic tissue response such as polyurethanes, silicones, and polyesters could be used and other polymers could also be used if they can be dissolved and cured or polymerized on the medical device such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose. The above polymers can be used to form a polymer matrix.

The polymer coatings or medical devices formed from polymeric material discussed herein may be designed with a specific dose of apolipoprotein. That dose may be a specific weight of apolipoprotein added or an apolipoprotein to polymer ratio. In one embodiment, the medical device can be loaded with about 1 to 1000 μg of apolipoprotein; in another embodiment, about 5 μg to 500 μg; in another embodiment about 10 μg to 250 μg; in another embodiment, about 15 μg 150 μg. A ratio may also be established to describe how much apolipoprotein is added to the polymer that is coated to or formed into the medical device. In one embodiment a ratio of 1 part apolipoprotein: 1 part polymer may be used; in another embodiment, 1:1-5; in another embodiment, 1:1-9; in another embodiment, 1:1-20.

In addition to the site specific delivery of apolipoproteins, the implantable medical devices discussed herein can accommodate one or more additional bioactive agents. The choice of bioactive agent to incorporate, or how much to incorporate, will have a great deal to do with the polymer selected to coat or form the implantable medical device or vise versa. A person skilled in the art can design medical devices for agent or agent combinations with immediate release, sustained release or a combination of the two.

Exemplary, non limiting examples of bioactive agents include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARy), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers described herein.

In another embodiment, a catheter based control release system is used to deliver apolipoproteins locally. Catheter systems are known in the art and can be used to deliver local treatment of a therapeutic agent or drug to a specific region of the body. There are several catheter based pump technologies known in the art which could be adapted for use with the polymers, bioactive agents and medical devices described herein. Herein, a catheter delivery system can be used to deliver an apolipoprotein, specifically ApoA I and/or ApoA IM to a specified region of the cardiovascular system.

In one embodiment, the catheter system comprises a mechanical system delivery system with one or more internal reservoirs for housing one or more bioactive agents. In one embodiment, the internal reservoirs can be mechanically refilled through the skin. The pump and reservoirs can be implanted subcutaneously in the abdominal region or any other place in the body wherein there is sufficient space to house the system. In one embodiment, the pump and reservoirs are at different locations in the body. In one embodiment, the system can be used to locally deliver the bioactive agent of interest locally to the site of interest.

More specifically, apolipoproteins can be delivered to the areas susceptible to and/or in need of treatment for stenosis, restenosis, ischemic myocardial infarct, atherosclerosis, stabilization of vulnerable plaque, and combinations thereof. Use of local treatment can help to reduce the effects of systemic administration as discussed supra.

### EXAMPLES

### Providing a Metallic Surface with an Apolipoprotein-Eluting Coating

The following Examples are intended to illustrate a non-limiting process for coating metallic stents with an apolipoprotein. One non-limiting example of a suitable metallic stent is the Medtronic/AVE S670™ 316L stainless steel coronary stent.

### Example 1

### Metal Stent Cleaning Procedure

Stainless steel stents were placed a glass beaker and covered with reagent grade or better hexane. The beaker containing the hexane immersed stents was then placed into an ultrasonic water bath and treated for 15 minutes at a frequency of between approximately 25 to 50 KHz. Next the stents were removed from the hexane and the hexane was discarded. The stents were then immersed in reagent grade or better 2-propanol and vessel containing the stents and the 2-propanol was treated in an ultrasonic water bath as before. Following cleaning the stents with organic solvents, they were thoroughly washed with distilled water and thereafter immersed in 1.0 N sodium hydroxide solution and treated at in an ultrasonic water bath as before. Finally, the stents were removed from the sodium hydroxide, thoroughly rinsed in distilled water and then dried in a vacuum oven over night at 40° C. After cooling the dried stents to room temperature in a desiccated environment they were weighed their weights were recorded.

### Example 2

### Coating a Clean, Dried Stent Using a Drug/Polymer System #1

In the following Example, the apolipoprotein is ApoA IM. Persons having ordinary skill in the art of polymer chemistry can easily pair the appropriate solvent system to the polymer-drug combination and achieve optimum results with no more than routine experimentation.

ApoA IM is carefully weighed and added to a small neck glass bottle containing water. The ApoA IM solution is then blended into a hydrophilic polymer. In this example, the polymer is dextran.

Dextran is slowly added to the ApoA IM solution and mixed until the dextran is dissolved, forming a drug/polymer solution.

The cleaned, dried stents are coated using either spraying techniques or dipped into the drug/polymer solution. The stents are coated as necessary to achieve a final coating weight of between approximately 10 μg to 1 mg. Finally, the coated stents are dried in a vacuum oven at 50° C. overnight. The dried, coated stents are weighed and the weights recorded.

The concentration of drug loaded onto (into) the stents is determined based on the final coating weight. Final coating weight is calculated by subtracting the stent's pre-coating weight from the weight of the dried, coated stent.

### Example 3

### Coating a Clean, Dried Stent Using a Drug/Polymer System #2

In the following Example, the apolipoprotein is ApoA IM. Persons having ordinary skill in the art of polymer chemistry can easily pair the appropriate solvent system to the polymer-drug combination and achieve optimum results with no more than routine experimentation.

ApoA IM is carefully weighed and added to a small neck glass bottle containing water. The ApoA IM solution is then blended into a hydrophilic polymer. In this example, the polymer is hyaluronic acid (HA).

HA is slowly added to the ApoA IM solution and mixed until the HA is dissolved, forming a drug/polymer solution.

The cleaned, dried stents are coated using either spraying techniques or dipped into the drug/polymer solution. The stents are coated as necessary to achieve a final coating weight of between approximately 10 μg to 1 mg. Finally, the coated stents are dried in a vacuum oven at 50° C. overnight. The dried, coated stents are weighed and the weights recorded.

The concentration of drug loaded onto (into) the stents is determined based on the final coating weight. Final coating weight is calculated by subtracting the stent's pre-coating weight from the weight of the dried, coated stent.

### Example 4

### Coating a Clean, Dried Stent Using a Sandwich-Type Coating

A cleaned, dry stent is first coated with HA or another suitable polymer followed by a coating of ApoA IM. Finally, a second coating of HA is provided to seal the stent thus creating a HA-ApoA IM-HA sandwich coated stent. The Sandwich Coating Procedure:

HA is added to an Erlenmeyer containing water. The flask is carefully mixed until all of the HA is dissolved. In a separate clean, dry Erlenmeyer flask ApoA IM is added to water and mixed until dissolved.

A clean, dried stent is then sprayed with HA until a smooth confluent polymer layer was achieved. The stent was then dried in a vacuum oven at 50° C. for 30 minutes.

Next, successive layers of ApoA IM are applied to the polymer-coated stent. The stent is allowed to dry between each of the successive ApoA IM coats. After the final ApoA IM coating has dried, three successive coats of HA are applied to the stent followed by drying the coated stent in a vacuum oven at 50° C. over night. The dried, coated stent is weighed and its weight recorded.

The concentration of drug in the drug/polymer solution and the final amount of drug loaded onto the stent determine the final coating weight. Final coating weight is calculated by subtracting the stent's pre-coating weight from the weight of the dried, coated stent.

### Example 5

### Coating a Clean, Dried, Porous Stent with Pure Drug

ApoA IM is carefully weighed and added to a small neck glass bottle containing water. The ApoA IM solution is then heated at 50° C. for 15 minutes and then mixed until the apolipoprotein is completely dissolved.

Next a clean, dried, porous stent is then sprayed with, or in an alternative embodiment, dipped into, the ApoA IM solution. The porous filled stent is dried in a vacuum oven at 50° C. over night. The dried, coated stent was weighed and its weight recorded.

The concentration of drug loaded into the pores of the stents is determined based on the final coating weight. Final coating weight is calculated by subtracting the stent's pre-coating weight from the weight of the dried, coated stent.

### Example 6

### Abdominal Aneurysm

In one embodiment, a stent loaded with at least one of ApoA I or ApoA IM can be used to deliver the apolipoprotein locally to the abdominal aorta for treatment/stabilization of an abdominal aneurysm.

### Example 6

### Local Delivery to Coronary Artery

In one embodiment, a stent loaded with at least one of ApoA I or ApoA IM can be used to deliver the apolipoprotein locally to the coronary artery for the combined treatment of restenosis and atherosclerotic plaque stabilization.

### Example 7

### Catheter Delivery to Abdominal Aneurysm

In one embodiment, a catheter delivery system loaded with at least one of ApoA I or ApoA IM can be used to deliver the apolipoprotein locally to the abdominal aorta for treatment/stabilization of an abdominal aneurysm.

### Example 8

### Catheter Delivery to Coronary Artery

In one embodiment, a catheter delivery system loaded with at least one of ApoA I or ApoA IM can be used to deliver the apolipoprotein locally to the coronary artery for the combined treatment of restenosis and atherosclerotic plaque stabilization.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A medical device system for localized treatment of a cardiovascular condition comprising:
a medical device;
a polymer matrix associated with said medical device; and
a therapeutically effective amount of an apolipoprotein, said apolipoprotein residing within said polymer matrix.

2. The medical device system according to claim 1, wherein said apolipoprotein is ApoA-I Millano.

3. The medical device system according to claim 2, wherein said ApoA-I Millano is present at a weight of 1 μg to 1000 μg.

4. The medical device system according to claim 1, wherein said apolipoprotien is ApoA-I peptide mimetic.

5. The medical device system according to claim 4, wherein said ApoA-I peptide mimetic is present at a weight of 1 μg to 1000 μg.

6. The medical device system according to claim 1, wherein said medical device is selected from the groups consisting of stents, catheters, micro-particles, probes, and vascular grafts.

7. The medical device system according to claim 1, wherein said cardiovascular condition is selected from the group consisting of plaque rupture, aneurysm, stenosis, restenosis, atherosclerosis, ischemic myocardial infarct, and combinations thereof.

8. The medical device system according to claim 1, wherein said polymer matrix is biodegradable.

9. The medical device system according to claim 8, wherein said polymer matrix comprises polymers selected from the group consisting of poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes and biomolecules such as fibrin, fibrinogen, cellulose, polysaccharides or carbohydrates (i.e. starch, hyaluronic acids, dextran, heparin sulfate, chondoritin sulfate, heparin, alginate), proteins (i.e. polyamino alcohols, polyphosphazines, polyanhidrides), collagen, and combinations thereof.

10. The medical device system according to claim 1, wherein said medical device comprises a ratio of polymer to apolipoprotein, wherein said ratio is between about 1:1 to about 1:20.

11. A method for localized treatment of a cardiovascular condition comprising:
a) providing a medical device comprising a polymer matrix, wherein said polymer matrix comprises a therapeutically effective amount of an apolipoprotein, said apolipoprotein;
b) implanting said medical device within a blood vessel; and
c) allowing said medical device to locally deliver said apolipoprotein thereby treating said cardiovascular condition.

12. The method according to claim 11, wherein said apolipoprotein is ApoA-I Millano.

13. The method according to claim 12, wherein said ApoA-I Millano is present at a weight of 1 μg to 1000 μg.

14. The method according to claim 11, wherein said apolipoprotien is ApoA-I peptide mimetic.

15. The method according to claim 14, wherein said ApoA-I peptide mimetic is present at a weight of 1 μg to 1000 μg.

16. The method according to claim 11, wherein said medical device is selected from the groups consisting of stents, catheters, micro-particles, probes, and vascular grafts.

17. The method according to claim 11, wherein said cardiovascular condition is selected from the group consisting of plaque rupture, aneurysm, stenosis, restenosis, atherosclerosis, ischemic myocardial infarct, and combinations thereof.

18. The method according to claim 11, wherein said polymer matrix is biodegradable.

19. The method according to claim 18, wherein said polymer matrix comprises polymers selected from the group consisting of poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes and biomolecules such as fibrin, fibrinogen, cellulose, polysaccharides or carbohydrates (i.e. starch, hyaluronic acids, dextran, heparin sulfate, chondoritin sulfate, heparin, alginate), proteins (i.e. polyamino alcohols, polyphosphazines, polyanhidrides), collagen, and combinations thereof.

20. The method according to claim 11, wherein said medical device comprises a ratio of polymer to apolipoprotein, wherein said ratio is between about 1:1 to about 1:20.
